(19) United States
(12) Patent Application Publication
(10) Pub. No.: US 2004/0093623 A1
(43) Pub. Date: May 13, 2004

(54) Non-human animal disease models

(76) Inventors: Zeger Debyser, Heverlee (BE); Erwin Lauwers, Zaventem (BE); Bart Nuttin, Rotselaar (BE); Veerle Baekelandt, Heverlee (BE); Bart De Strooper, Hoeilaart (BE)

Correspondence Address: CLARK & ELBING LLP, 101 FEDERAL STREET, BOSTON, MA, 02110, US

(21) Appl. No.: 10/450,873

(22) PCT Filed: Dec. 18, 2001
(86) PCT No.: PCT/BE01/00216

(30) Foreign Application Priority Data
Dec. 20, 2000 (GB) 0031054.0
Oct. 30, 2001 (GB) 0126021.5

(51) Int. Cl. A01K 67/027

(52) U.S. Cl. 800/9

(57) ABSTRACT

The invention involves a method of stereotactic and viral vector-mediated gene transfer to produce animals harboring in their neural tissue a polynucleotide sequence, an allelic variant, minigene or a homolog thereof, that encodes for α-synuclein or functional homnologues thereof and overexpresses ccsynuclein or functional homologues thereof locoregional in said neural tissue. Overexpression of α-synuclein is associated with locoregional pathology in the neural tissue as evidenced by histology and neurodegeneration as evidenced by histology. These animals can be used in pharmaceutical screening and for in vivo modelling of α-synuclein biochemistry.

### TECHNICAL FIELD

The invention provides non-human animals harboring in their neural tissue a polynucleotide sequence, an allelic variant, minigene or a homologue thereof, that encodes for α-synuclein or functional homologues thereof and overexpresses α-synuclein or functional homologues thereof locoregional in said neural tissue the over-expression resulting in neuropathology and/or neurodegeneration. The invention also provides targeting constructs used to produce such non-human animals with locoregional neural transgenes, and methods for using the non-human animals with locoregional neural transgenes in pharmaceutical screening and for modelling neurodegenerative diseases (e.g., Parkinson disease) and for in vivo modelling of α-synuclein biochemistry. Furthermore the invention comprises a method for reducing the immunogenicity of the lentiviral vector compositions, used for lentiviral mediated gene transfer in the brains of non-human animals.

### BACKGROUND OF THE INVENTION

Parkinson's Disease

Parkinson's disease (PD) is a progressive neurodegenerative disease, diagnosed by a triad of clinical symptoms: muscle rigidity, tremor and bradykinesia. Old age is the single most important risk factor. The prevalence of PD in Europeans is 1.6% in persons over 65 years of age. Some 50 000 Americans each year are diagnosed with Parkinson's disease. Worldwide, there were an estimated 4 million people suffering from the disease in 1990. More than 10% of the patients however, are diagnosed before the age of 50. The disease is characterised by the progressive degeneration of dopaminergic neurons in the substantia nigra pars compacta. Symptoms appear when 80% of these neurons are lost. The presence of Lewy bodies, eosinophilic inclusions in the cytoplasm of nigral cells, is generally considered to be a neuropathological hallmark of the disease. Although the role ofenvironntal factors has always been emphasised in PD, it is now recognised that there is a genetic component in some familial forms. The following genes have been associated with PD. At least two missense mutations in α-synuclein, the main constituent of Lewy bodies, namely A30P and A53T have been identified in familial PD (Polymeropoulos et al., 1997; Kruger et al., 1998). Interestingly, α-synuclein, a presynaptic phosphoprotein, was also identified as the non-amyloid component of plaques in AD (Ueda et al., 1993). It has been shown in vitro that mutant forms of the soluble presynaptic protein α-synuclein oligomerize more readily (Conway et al., 2000). Ablation of the gene encoding α-synuclein in mice results in functional deficits of the nigrostriatal dopamine system (Abeliovich et al., 2000) providing further evidence for a connection of α-synuclein with PD. Another protein, parkin, was shown to be mutated in an autosomal recessive form of juvenile parkinsonism without Lewy body formation (Kitada et al., 1998). Two further genes have been identified, the thiol protease UCH-L1 (Leroy et al., 1998), and one on chromosome 4 (Farrer et al., 1998), while another gene, PARK3, has been suggested to be involved in PD (Gasser et al., 1998).

Animal Models for PD

The classical animal models for PD have been developed in rat and monkey. The most widely used model involves stereotactic injection of neurotoxic 6-hydroxydopamine in the nigrostriatal pathway of rats, which leads to unilateral degeneration of the nigral dopaliinergic neurones. Behaviourally this can be tested by apomoiphine- or amphetamine-induced rotational asymmetry. Injection into the terminal field of nigral dopaminergic neurones, which causes a progressive degeneration of these cells (Sauer and Oertel, 1994), appears to be the most relevant variant of this PD model. A more elaborate disease model in non-human primates has been developed by chronic IV injection of MPTP (Burns et al., 1983). The MPTP model in monkeys replicates most of the clinical signs of PD in human patients, but is less accessible because of financial, infrastructural and bioethical concerns. It was recently reported that chronic systemic administration of the pesticide rotenone to rats reproduced some of the neuropathological, neurochemical and behavioural characteristics of PD (Betarbet et al. 2000).

The identification of α-synuclein as a genetic component in PD has very recently lead to the development of transgenic animal models in mouse and Drosophila. Neuronal overexpression of wild-type α-synuclein in mice led to cytoplasm and nuclear inclusion body formation containing α-synuclein (Masliah et al., 2000). The transgenic non-human animals also display dopaminergic deficits, more specifically decreases in tyrosine hydroxylase-positive nerve terminals and in activity of the enzyme in the striatum. Furthermore, behavioural tests revealed locomotor impairments in these mice. However, some cardinal features of PD have yet to be observed in the α-synuclein transgenic mice. There is no loss of dopamine neurones and the inclusions do not contain fibrils characteristic of Lewy bodies (Dawson, 2000b; Haass and Kahle, 2000). Overexpression of wild type and mutant α-synuclein in the fruit fly reproduces strikingly many features of PD (Feany and Bender, 2000). There is an age-dependent specific loss of dopamine-secreting neurones starting at mid-life. Some neurones accumulate intracellular aggregates containing α-synuclein fibrils that closely resemble Lewy bodies. Moreover, the flies display a progressive, age-dependent loss of motor function. Although the fruit fly model resembles PD at first sight and enables genetic studies, drug screening and gene therapy are more relevant in rodents. These first reports on α-synuclein transgenic animals not only confirm the importance of α-synuclein in PD, but also stress the need for even better experimental animal models, that will allow further research on the pathogenesis of the disease and on new therapeutic targets and strategies (Dawson, 2000a).

Classical transgenesis has several disadvantages: 1) Overexpression or knocldng out of some proteins may be lethal at the embryonic age 2) Alternative pathways may be induced to compensate for the missing protein or to counteract the new protein 3) Even when using tissue-specific promoters, locoregional distribution of expression of the genes of interest is difficult to obtain 4) Except when using inducible promoters (that rarely work), protein overexpression cannot be initiated at old age. The latter may be important to induce neurodegeneration 5) Transgenics are mainly restricted to mice; they may not always provide the relevant disease model 6). Making transgenic mice is a labor-intensive and time-consuming activity. The results are often unpredictable. For each variant a new mouse has to be created. Between the conceptual phase and data collection one and a half to two years will have elapsed and 7) Each mouse is made in a particular genetic background. This may induce a specific bias.

Thus, there is a need in the art for non-human animals harboring intact disease-associated gene(s), that encode(s) for disease related proteins, factors or markers in selected or targeted neural tissue.

Present invention demonstrates that by stereotactic vector-mediated transfer of disease-associated genes into the brain (or another organ) overexpression of pathogenic peptides, polypeptides, proteins, markers or fragments thereof can be achieved and animals with locoregional neural transgenes can be created. By using antisense, transdominant negative mutant or ribozyme technology, one can downregulate proteins involved in pathogenetic pathways. If the gene transfer results in pathology reminiscent of the disease, a disease model has been created.

The method of present invention has the following advantages: 1. The embryonic age and potential lethality associated with the transgenes can be avoided. 2. Compensatory changes in gene expression during embryogenesis cannot occur. 3. Locoregional distribution of expression of the genes is obtained by stereotactic injection. 4. Protein overexpression can be initiated at different ages, especially old age. The latter may be important to induce neurodegeneration. Injections can also be repeated. 5. Once the vector is cloned, locoregional transgenics can be created in different species to provide for the relevant disease model (mouse, rat, monkey). 6. Results can be obtained in a couple of months. Time depends on cloning and expression time and 7. The experiments can be readily reproduced in different background strains.

### ILLUSTRATIVE EMBODIMENT OF THE INVENTION

In general, the invention features, a method to create disease models in non-human animals or to create anmals with locsregional tansgenes by stereotactic and viral vector-mediated gene transfer. It also involves a method to create locoregional, somatic transgenic non-human animals using vector-mediated gene transfer.

Illustrative Embodiments of the Methods

In its method aspect this invention concerns the creation by stereotactic lentiviral vector mediated gene transfer of non-human animals with locoregional transgenes or of disease models in non-human animals. More preferably this method involves stereotactic lentiviral vector mediated gene transfer in he brain of various non-human animal species. In its most preferred embodiment the method of this invention involves stereotactic lentiviral vector mediated gene transfer in the brain of rodents (mice, rat).

Yet another method aspect of the invention is a method to create animals with locoregional transgenes or to create disease models in non-human animals using stereotactic lentiviral vector mediated gene transfer in the brain to overexpress or prevent expression of disease associated genes involved in neurodegeneration to develop neuropathologies.

Yet another method aspect the invention is the creation of animals with locoregional transgenes or of disease models in non-human animals by locoregional transgenes using stereotactic lentiviral vector mediated gene transfer in the brain to overexpress or prevent expression of disease associated genes involved in Parkinson's disease. In one embodiment this involves a method to create disease models in non-human animals or to create animals with locoregional transgenes using stereotactic lentiviral vector mediated gene transfer in the brain to overexpress wild type or mutant α-synuclein or functional derivatives thereof.

Yet another aspect of present invention is the visualisation by histological stains, histochemical stains or immunohistochemical stains the neurological patholgies. These histological pathologies can be characterised by a technology of the group consisting of electron micropys, transmission electron microscope, phase-contrast microscope, scanning electron microscope, transmission microscopy and light microscopy.

Another approach of visualisation of the neurological pathologies is radiographic or magnetic neuroimaging. Radiographic neuroimaging modalities such as Computed Tomography (CT) and magnetic neuroimaging modalities such us Magnetic Resonance Imaging (MRI) and improved MRI technologies such as 3D-MRI, gadolinium (Gd)-enhanced MRI and Magnetic Resonance Spectroscopic Imaging (MRSI) and Microscopic Magnetic Resonance Imaging (IMRI) supported by quantitative analysis and comparison of the structure in neuroimages obtainable by these modalities, are available for screening of neurodegenerative pathologies. The diagnostic technique that provides high-quality cross-sectional images of the brains provides fast, painless, and non-invasive ways to distinguishing of normal and traumatised tissues and in particular, the exquisite image contrast and spatial detail of MR images has led to a general use of these magnetic neuroimaging modalities in patients with a variety of suspected neurological diseases. For instance, Lewy bodies and dystrophic neurites being considered a common substrate for dementia, the primary component of this pathology involving α-synuclein, are distinguishable by microscopic magnetic resonance imaging (μMRI) techniques, while white matter changes, having an increased frequency in Alzheimer's disease (AD) and subcortical vascular dementia (SVD), can be visualised by CT or MRI of the brain. The data on the neuorimages obtainable by MRI can be processed by tissue segmentation procedures to quantify gray matter, white matter, cerebrospinal fluid, lacunes, atrophy and white matter lesions to allow accurate diagnoses and assessment of neurodegenerative pathologies. MRI slices can be used to make volumetric measurements of the brains and these data can be manipulated and rendered into 3D to give dramatic pictures of specific brain regions.

Illustrative Embodiments of the Non-human Animal Models

In accordance with the foregoing objects, in one aspect of the invention are provided non-human animals harboring in their neural tissue or brains a polynucleotide sequence, an allelic variant or a homologue thereof, that encodes for α-synuclein or functional homologues thereof and overexpresses α-synuclein, isoform of α-synuclein or functional homologues thereof locoregional in said neural tissue or said brains.

The invention further provides non-human animals with locoregional transgenic brains, typically non-human mammals such as mice, which harboring in their neural tissue a polynucleotide sequence, an allelic variant or a homologue thereof, that encode for α-synuclein or functional homologues thereof and overexpresses α-synuclein or functional homologues thereof locoregional in said neural tissue.

One aspect of this invention involves a non-human animal model for in vivo modelling of the of α-synuclein biochemistry based on local, viral vector-mediated overexpression of wild type or mutant α-synuclein or functional derivatives thereof. More preferably the invention involves an animal model in mice for in vivo modelling of the α-synuclein biochemistry based on local, viral vector-mediated overexpression of wild type or mutant α-synuclein.

Another preferred embodiment is a non-human animal model for Parkinson's disease based on local, viral vector-mediated overexpression of wild type or mutant α-synuclein or functional derivatives thereof. Most preferably the invention involves an animal model in mice for Parkinson's disease based on local, viral vector-mediated overexpression of wild type or mutant α-synuclein or functional derivatives thereof.

The invention also provides transgenes comprising the α-synuclein gene an allelic variant, minigene, a homolog thereof or A30P, that encode for α-synuclein, an isoform of α-synuclein or functional homologues thereof or at least a portion thereof, said transgene obtainable by a method comprising 1) producing HIV-1-derived vector particles, pseudotyped with the envelope of non related virus, said HIV-1 derived vector particles obtainable by tranfecting suitable cells in suitable agents with a suitable packaging plasmid encoding viral gag and pol proteins, a plasmid encoding the envelope of a non related vrus andua plasmid encoding α-synuclein gene or an allelic variant, minigene or a homolog thereof which is flanked by LTR's, 2) isolating and concentrating the vector particles 3) redisolving the vector particles in a suitable agent, 4) injecting the vector particle solution in stereotactically defined targets of an non-human animal brain.

The invention also provides transgenes comprising the α-synuclein gene an allelic variant, minigene, a homolog thereof or A30P, that encode for α-synuclein, an isoform of α-synuclein or functional homologues thereof or at least a portion thereof, said transgene obtainable by a method comprising producing HIV-1-derived vector particles, pseudotyped with the envelope of non related virus, said HIV-1 derived vector particles obtainable by tranfecting suitable cells in suitable agents with a suitable packaging plasmid encoding viral gag and pol proteins, a plasmid encoding the envelope of a non related virus and the α-synuclein encoding plasmid, pHMWS-SYN(α-synucleingene)-h), 2) isolating and concentrating the vector particles 3) redisolving the vector particles in a suitable agent, 4) injecting the vector particle solution in sterotactically defined targets of an non-human animal brain.

The invention also provides transgenes comprising the α-synuclein gene an allelic variant, minigene, a homolog thereof or A30P, that encode for α-synuclein, an isoform of α-synuclein or functional homologues thereof or at least a portion thereof, said transgene obtainable by a method comprising producing HIV-1-derived vector particles, pseudotyped with the envelope of non related virus, said HIV-1 derived vector particles obtainable by tranfecting suitable cells in suitable agents with a suitable packaging plasmid encoding viral gag and pol proteins, a plasmid encoding the envelope of a non related virus and the α-synuclein encoding plasmid, pHMWS-SYN(α-synucleingene)-h) (See FIG. 1), a derivative of pHR′ (Naldini et al., 1996)2) isolating the vector particles 3) redisolving the vector particles in a suitable agent, 4) injecting the vector particle solution in sterotactically defined targets of an non-human animal brain 5) histochemical analysis of expression of transgene and induction of pathology.

The present invention also comprises cell lines derived from SKNSH: neuroblastoma cells that overexpress wild type, mutant or antisense α-synuclein and were elected after transduction with the respective lentiviral vector. These cell lines can be used for evaluating the effect of pharmaceutical compositions including nucleic acids on synucleopathy.

The present invention further comprises administering (systemically or locally) pharmaceutical compositions in a pharmaceutically acceptable carrier in the created animal models and/or cell lines and verifying whether the compound alters expression of transgene qualitatively or quantitatively and/or alters the observed pathology. Such pharmaceutical compositions should contain a therapeutic amount of at least one compound identified by the method of the present invention. Such compound may be a nucleic acid encoding a protein or fragment of a protein. The pharmaceutically acceptable carrier can be any compatible, non-toxic substance suitable to deliver the compounds to an intended animal host or cell line. Sterile water, alcohol, fats, waxes, and inert solids may be used as the carrier. Pharmaceutically acceptable adjuvants, buffering agents, dispersing agents, and the like may also be incorporated into the pharmaceutical compositions. Preparation of pharmaceutical conditions incorporating active agents is well described in the medical and scientific literature. See, for example, Remington's Pharmaceutical Sciences, Mack Publishing Company, Easton, Pa., 16th Ed., 1982, the disclosure of which is incorporated herein by reference.

The pharmaceutical compositions just described are suitable for injection in targeted zones and regions of the brain or neural tissue systemic administration to the host, including both parenteral, topical, and oral administration. The pharmaceutical compositions may be administered parentally, i.e. subcutaneously, intramuscularly, or intravenously. Thus, the present invention provides compositions for administration to an animal host, where the compositions comprise a pharmaceutically acceptable solution of the identified compound in an acceptable carrier, as described above.

### BRIEF DESCRIPTION OF THE FIGURES

FIG. 1 pHMWS is a derivative of pHR′ (see reference Naldini et al, 1996) and pHMWS-SYN WT contains the gene for human a-syncuclein wild type, pHMWS-SYNA30P contains the gene for human α-synuclein, with the A30P mutation (Kruger et al., 1998; Polymeropoulos et al., 1997).

FIG. 2 Stable cell lines that express wild type or (A30P) α-synuclein in cell culture after LV transduction. a, Cell lysates from SKNSH cells after transduction with vectors encoding wild type, mutant and antisense α-synuclein. b, α-synuclein staining revealed high expression levels of wild type and mutant α-synuclein in stable cell lines. c, Immunocytochemical staining of the stable cell line overexpressing the (A30P) mutant α-synuclein with an α-synuclein antibody.

FIG. 3. Two weeks after lentiviral vector pBMWS-SYNA30P mediated gene transfer in mouse striatum α-synuclein overexpression is demonstrated by immunohistochemistry. Most of the transduced cells have a neuronal morphology(a). Overexpression was also evidenced after LV delivery into the substantia nigra (b) and the striatum (c) of the rat. Scale bar in a, b and c represent 100 μm, 50 μm and 20 μm respectively.

FIG. 4. New protocol for vector production.

FIG. 5. Lentiviral vectors were prepared and purified following different procedures. Aliquots of vector were boiled, separated by electrophoresis and silver stained. It is clear that serum free production and sucrose gradient purification result in more pure vector preparations.

FIG. 6. Vector performance All vectors were produced in 293T cells using following plasmids: transfer plasmid pH GFP W 20μg per plate; packaging plasmid p8.91 10 μg and envelop plasmid pMDG 5μg

FIG. 7. Immune response in mouse serum to vector proteins is prevented partially by production of vectors without serum (serum free) and prevented completely by purification of concentrated vector preps by sucrose gradient ultracentrifugation (sucrose).

FIG. 8. In vivo expression of EGFP after serum free production of vectors. Immunohistochemistry for EGFP 2 weeks after stereotactic injection of concentrated HIV vector (pCHWSEGFP) into the mouse striatum. One hemisphere was injected with vector produced according to the normal procedure; the other side was injected with a serum free vector batch normalised for p24.

FIG. 9. No decrease in NeuN expression after serum free production of vectors or after sucrose gradient purification. (Upper panels) Immunohistochemistry for EGFP 2 weeks after stereotactic injection of concentrated HIV vector (pCHWSEGFP) into the mouse striatum. Mice were injected with normalised vector preps made according to the different procedures (Lower panels) Adjacent sections were stained for the neuronal marker NeuN.

FIG. 10. Neuropathology associated with overexpression of wild type and mutant α-synuclein in mouse striatum two weeks after gene transfer. a, Human wild type α-synuclein containing neurites with a beaded morphology. b, Higher magnification of these neuritic enlargements. Scale bars represent 5 μm.

FIG. 11. Neuropathology is more pronounced after longer time intervals. a, At 6 months postinjection we observed small cytoplasmic α-synuclein containing inclusion bodies. b, At 10-12 months post injection α-synuclein overexpressing neurons form cytoplasmic inclusion bodies that are often large and spherical. c, At this time point the observed cytoplasmic inclusions were occasionally tangle-like. d, Double immunofluorescent stainings demonstrating the presence of α-synuclein (green) and ubiquitin (red) positive cytoplasmirc inclusions 6 months postinjection. e, At 10-12 months post injection the x-synuclein expressing neurons (green) contained very large ubiquitin-positive cytoplasmic inclusions (red). f, The neuritic enlargements also colocalise for α-synuclein (green) and ubiquitin (red). Scale bars represent 5 μm.

FIG. 12. Number of transduced cells at different time points after gene delivery. α-synuclein positive cells in the striatum were counted at different time points after injection of LV encoding the wild type or mutant α-synuclein. The total number of transduced cells at two weeks was set at 100%. There is a significant difference between the number of cells expressing α-synuclein at 10-12 months compared with 2 weeks (P<0.05) and with 6 months (P<0.05).

FIG. 13. Neurodegeneration in the mouse striatum after overexpression of wild type and mutant α-synuclein. a, At 10-12 months post injection several Fluoro-Jade B-positive cells and fibers were detected after injection of wild-type or mutant α-synuclein. b, In control treated animals we detected no such staining pattern. Scale bars represent 100 μm.

FIG. 14. Neurodegeneration in rodent brain upon α-synuclein overexpression a and b, Several degenerating neurons in brain sections can be double-stained for α-synuclein (green) and ubiquitin (red). Scale bars represent 5 μm.

### DEFINITIONS

Generally, the nomenclature used hereafter and the laboratory procedures in cell culture, molecular genetics, and nucleic acid chemistry and hybridisation described below are those well known and commonly employed in the art. Standard techniques are used for histology, polynucleotide synthesis, cell culture and recombinant DNA technology (Ausubel, F. M., Brent, R., Kingston, R. E., Moore, D. D., Seidman, J. G., Smith, J. A., Struhl, K Eds. (1987) Current protocols in molecular biology John Wiley & Sons New York).

Generally, enzymatic reactions oligonucleotide synthesis, and purification steps are performed according to the manufacturer's specifications. The techniques and procedures of stereotactic surgery are generally performed according to conventional methods in the art and various general references which are provided throughout this document. The procedures therein are believed to be well klown in the art and are provided for the convenience of the reader. All the information contained therein is incorporated herein by reference. Unique technologies are detailed and explained in the examples.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of the present invention, the following terms are defined below.

The term “non-dividing cell” used herein means animal cells such as nerve, liver, muscle and bone marrow stem cells

The term “α-synuclein gene ” means a α-synuclein gene an allelic variant, minigene, a homolog thereof or A30P, that encode for α-synuclein, an isoform of α-synuclein or functional homologues thereof or at least a portion thereof

The term “Transgene” means any piece of DNA which can bee inserted into a cell, and preferably becomes part of the genome of the resulting organism (i.e. either stably integrated or as a stable extrachromosomal element). Such a transgene includes genes which are partly or entirely heterologous (i.e. foreign) as well as genes homologous to endogenous genes of the organism. Including within this definition is a transgene created by providing an RNA sequence with is reverse transcribed into DNA and then incorporated into the genome, or an antisense agent or molecule.

The term “animal” herein is used to mention non-human animals

The term “transgenic animal” is used herein to mention non-human animals, having a non-endogenous (i.e. heterologous) nucleic acid sequence present as a extrachromosomal element in stably integrated into its germ line DNA (i.e. in the genomic DNA of most or all of its cells). Heterologous nuleic acid is introduced into the germ line of such transgenic animals by genetic manipulation of, for example, embryous or embryonic stem cells of the host animal. A “transgenic” animal is any animal containing cells that bear genetic information received, directly or indirectly, by deliberate genetic manipulation at the subcellular level

The term “animals with locoregional neural transgenes” is used herein to mean non-human animals which overexpresses a exogenous peptide, polypeptide or protein or at least a portion thereof in at least one precisely localised region in the brain or other neural tissue after local delivery, preferably stereotactic vector-mediated transfer, of a “heterologous gene” or “heterologous polynucleotide sequence” encoding a exogenous peptide, polypeptide or protein or at least a portion thereof.

The term “locoregional transgenic brains” is used herein to mean brains of non-human animals which overexpresses a exogenous peptide, polypeptide or protein or at least a portion thereof in at least one precisely localised region in the brain or other neural tissue after local delivery, preferably stereotacted vector-mediated transfer, of a “heterologous gene” or “heterologous polynucleotide sequence” encoding a exogenous peptide, polypeptide or protein or at least a portion thereof.

The term “vector” is used herein to mean that a DNA molecule, derived, e.g., from a plasmid or virus, into which fragments of DNA may be inserted or cloned. A vector will contain one or more unique restriction sites and may be capable of autonomous replication in a defined host or vehicle organism such that the cloned sequence is reproducible.

The term “antisense agent” refers to a molecule which interacts directly with intracellular DNA or RNA to achieve a therapeutic effect. Examples of antisense agents include, without limitation, DNA-binding molecules, triple-helix (or triplex) forming agents, ribozymes, and the like. Antisense agents may be prepared from naturally-occurring nucleotides, or may contain modified bases.

As used herein, a “heterologous gene” or “heterologous polynucleotide sequence” is defined in relation to the animals with locoregional neural transgenes producing such a gene product in targeted zones or regions of the brain or neural tissue. A heterologous polypeptide, also referred to as a xenogenic polypeptide, is defined as a polypeptide having an amino acid sequence or an encoding DNA sequence corresponding to that of a cognate gene found in an organism not consisting of the animals which harbored the locoregional neural transgenes. Thus, an ″ animal with locoregional expression in brains or neural tissue of a α-synuclein gene can be described as harbouring a heterologous α-synuclein gene. A cognate heterologous gene refers to a corresponding gene from another species; thus human α-synuclein is a cognate heterologous gene for mice. A mutated endogenous gene sequence can be referred to as a heterologous gene.

As used herein, the term “minigene” refers to a heterologous gene construct wherein one or more nonessential segments of a gene are deleted with respect to the naturally-occurring gene. Typically, deleted segments are intronic sequences of at least about 100 basepairs to several kilobases, and may span up to several tens of kilobases or more. Isolation and manipulation of large. (i.e., greater than about 50 kilobases) targeting constructs is frequently difficult and may reduce the efficiency of transferring the targeting construct into a host cell. Thus, it is frequently desirable to reduce the size of a targeting construct by deleting one or more nonessential portions of the gene. Typically, intronic sequences that do not encompass essential regulatory elements may be deleted. Frequently, if convenient restriction sites bound a nonessential intronic sequence of a cloned gene sequence, a deletion of the intronic sequence may be produced by: (1) digesting the cloned DNA with the appropriate restriction enzymes, (2) separating the restriction fragments (e.g., by electrophoresis), (3) isolating the restriction fragments encompassing the essential exons and regulatory elements, and (4) ligating the isolated restriction fragments to form a minigene wherein the exons are in the same linear order as is present in the germline copy of the naturally-occurring gene. Alternate methods for producing a minigene will be apparent to those of skill in the art (e.g., ligation of partial genomic clones which encompass essential exons but which lack portions of intronic sequence). Most typically, the gene segments comprising a minigene will be arranged in the same linear order as is present in the germline gene, however, this will not always be the case. Some desired regulatory elements (e.g., enhancers, silencers) may be relatively position-insensitive, so that the regulatory element will function correctly even if positioned differently in a minigene than in the corresponding germline gene. For example, an enhancer may be located at a different distance from a promoter, in a different orientation, and/or in a different linear order. For example, an enhancer that is located 3′ to a promoter in germline configuration might be located 5′ to the promoter in a minigene. Similarly, some genes may have exons which are alternatively spliced at the RNA level, and thus a minigene may have fewer exons and/or exons in a different linear order than the corresponding germline gene and still encode a functional gene product. A cDNA encoding a gene product may also be used to construct a minigene. However, since it is often desirable that the heterologous minigene be expressed similarly to the cognate naturally-occurring non-human gene, transcription of a cDNA minigene typically is driven by a linked gene promoter and enhancer from the naturally-occurring gene. Frequently, such minigene may comprise a transcriptional regulatory sequence (e.g., promoter and/or enhancer) that confers neuron-specific or CNS-secific transcription of the minigene α-synuclein encoding sequences.

The term “agent” is used herein to denote a chemical compound, a mixture of chemical compounds, a biological macromolecule, or an extract made from biological materials such as bacteria, plants, fungi, or animal particularly mammalian) cells or tissues.

As used herein, “isoform α-synuclein” and “α-synuclein isoform” refer to a (poly)peptide or protein that is encoded by at least one exon of the α-synuclein gene

### MATERIAL AND METHODS

Animals

Female C57BL/6 mice and Wistar rats of 8 weeks old were used in this study. The animals were housed under 14 h light/10 h dark cycle with free access to food and water.

Lentiviral Vector Production

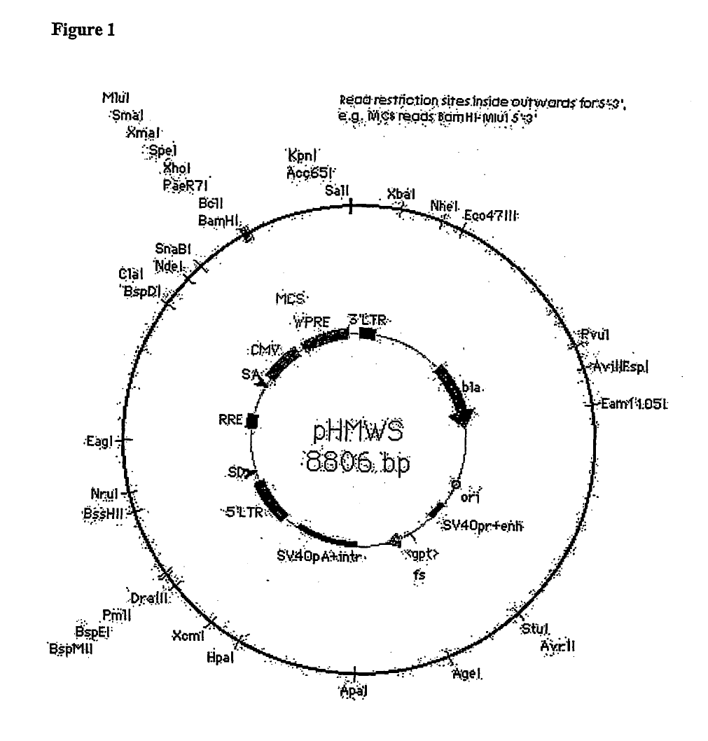

HIV-1-derived vector particles, pseudotyped with the envelope of vesicular stomatitis virus (VSV), were produced by transfecting 293T cells (commonly available human embryonic kidney cell line) with a packaging plasmid encoding viral gag and pol proteins (pCMVAR8.91), a plasmid encoding the envelope of vesicular stomatitis virus (pMDG) and a plasmid encoding a human wild type α-synuclein or a clinical mutant of α-synuclein (A30P) flanked by two long terminal repeat sequences LTRs (pMWS-SYNh and pHMWS-SYN(A30P)h). Some experiments were carried out with pHR'-derived vectors not containing the WPRE element, that thus is not absolutely required for over-expression. All plasmids were kind gifts from Dr. O. Danos (Genethon, France) and Dr. D. Trono (Geneva, Switzerland) and are described in Naldini et al., 1996 and Zufferey et al., 1997. The construction of vector plasmid pHR' has been described by Verma et al, U.S. Pat. No. 6,013,516. The basic constiruct pMWS (FIG. 1) was constructed in our laboratory from the origina pHR' construct by including a multiple cloning site (MCS or M) behind the internal CMV promoter, the woodchuck hepatitis regulatory element (WPRE or W) and deletion of part of the LTR (SIN or S). The development of self-inactivating vectors (S), in which the viral promoter in the LTR is inactivated during reverse transcription, precludes oncogene activation and vector rescue (Zufferey et al., 1998). Finally, the posttranscriptional cis-acting regulatory element of the woodchuck hepatitis virus (W) has been inserted to increase transgene expression (Zufferey et al., 1999). The gene encoding α-synuclein wild type and (A30P) (Conway et al., 1998), was obtained from Kelly Conway (Center for Neurologic Diseases, Boston), and was cloned in the MCS of the pHMWS plasmid using SpeI and KpnI. Wild type and (A30P) α-synuclein were also cloned in the pHMIRESNeo transfer plasmid to create stable cell lines of SKNSH overexpressing both forms of α-synuclein. This transfer plasmid was constructed in our laboratory from the pHMS construct by including IRES and Neo elements.

Transient transfection of 293T cells was carried out in 10 cm dishes. Per plate a mixture (700 μl) containing 20 μg of vector plasmid, 10 μg of packaging construct and 5 μg of envelope plasmid was made in 150 mM NaCl. To this DNA solution 700 μl of a PEI solution (110 μl of a 10 mM stock solution in 150 mM NaCl) was added slowly. After 15 min at room temperature, the DNA-PEI complex was added dropwise to the 293T cells in DMEM medium with 1% FCS. After overnight incubation, medium was replaced with medium containing 10% FCS. Supernatants were collected from day two to five post-transfection, clarified by low speed centrifugation and filtered through a 0.45 μM filter. The vector particles were sedimented by ultracentrifmgation in a swinging-bucket rotor (SW27 Beckman, Palo alto, Calif.) at 25, 000 rpm for 2 hr at 4° C. Pellets were redissolved in PBS resulting in a 100-fold concentration. For injections into the brain, an additional centrifugation is performed in a fixed angle rotor (Biofuge Stratos, Heraeus Instruments, Hanau Germany) at 20, 000 rpm at 4° C. for 1 hr.

Surgery

All surgical procedures were performed under chloral hydrate anaesthesia (400 mg/kg i.p.) using aseptic procedures. The mice were placed in a stereotactic head frame (Narishige), and after midline incision of the skin, 1 or 2 small holes were drilled in the skull in the appropriate location using Bregma as reference. The coordinates used were: striatum mouse (AP 0.5, LAT 2.0, DV 3.0-2.0), substantia nigra rat (target AP -5.2, L 2.3, DV 7.3). Two μl of highly concentrated vector (10⁸ pg p24/ml) supplemented with 4 μg/ml polybrene was injected at a rate of 0.25 μl/min with a 30G needle connected by a microdialysis tubing to a 10 μl Hamilton syringe in a microinjection pump (CMA). In some animals, 2 μl of 0.9% NaCl solution was injected in the contralateral hemisphere as control. After the injection, the needle was left in place for an additional 10 minutes before being slowly withdrawn from the brain (adapted from Dull, et al., 1998).

Histology

To assess lentiviral transduction, the mice were deeply anaesthetised with pentobarbital and perfused transcardially with saline followed by ice-cold 4% paraformaldehyde in PBS for 15 minutes. The brain was removed from the skull and postfixed overnight in the same fixing solution. 50 μm thick coronal brain sections were cut with a vibratome and stored at 4° C. in PBS buffer containing 0.1% sodium azide. The sections were treated with 3% hydrogen peroxide and preincubated in 5% normal swine serum with 0.1% Triton X-100 in PBS. Incubation with the primary antibody in 5% normal swine serum and 0.1% Triton X-100 was overnight at room temperature. The sections were then incubated in biotinylated swine anti-rabbit secondary antibody, followed by an incubation with Strept-ABC-HRP complex (Dako, Glostrup, Denmark). Detection was with diaminobenzidine (DAB) using H₂O₂ as a substrate. For immunofluorescence, sections were incubated overnight with one or two primary antibodies. Detection was with a secondary biotinylated antibody followed by Texas Red-coniugated streptavidin (Jackson ImmunoRes Lab., West Grove, Pa.) and/or a FITC-conjugated secondary antibody (donkey anti-mouse, Jackson ImmunoRes Lab.; swine anti-rabbit, DAKO). Primary antibodies used were: rabbit polyclonal anti-α-synuclein (1:500-1:5000, Chemicon), mouse monoclonal anti-α-synuclein (1:20, ZYMED, South San Francisco, Calif.), rabbit polyclonal anti-ubiquitin (1:200, DAKO) and rabbit polyclonal anti-TH (1:1000, Chemicon). Analysis was done on a NIKON inverted microscope DIAPHOT 300 connected to a Bio-Rad MRC1024 confocal microscope and images were captured by Lasersharp (version 3.2) and processed using Adobe Photoshop 5.5 (Adobe, Calif.).

Fluoro-Jade B histochemistry was used to detect degenerating neurons according to the manufacturer's protocol (Histo-Chem, Jefferson, Ark.).

Cell Culture and Letiviral Vector Transduction

SK-N-SH cells (human neuroblastoma cells, ATCC HTB11) were maintained in Dulbecco's modified Eagle's medium (DMEM) with glutamax (Invitrogen, Belgium) containing 10% foetal calf serum (FCS), 0.12% (v/w) sodium carbonate (Invitrogen) and 20 μg/ml gentamycin (Invitrogen) in a 5% humidified CO₂ atmosphere. The day before transduction, we seeded the cells in a 6-well plate. On the day of transduction, medium was replaced by DMEM containing 1% FCS, 4μg/ml polybrene and 4×10⁶ pg p24 of LV. 5 hours after transduction, we replaced the medium and 2 days post-transduction we harvested the cells with PBS containing 5 mM EDTA. The cells were lysed with SDS containing 10 mnM PMSF (Sigma, Belgium) and boiled for 5 minutes. For the production of the stable cell lines we seeded SKNSH cells in a 24-well plate at a density of 100, 000 cells per well. Two days after transduction with 1×10⁶ pg p24 of pHMSYN(WT)IRESNeo or pHMSYN(A30P)IRESNeo vector , the medium was replaced with medium containing 800 μg/ml geneticine (G418, GICO-BRL). Analysis of expression was performed after 2 weeks of selection.

Western Blot Analysis

10 μg total protein was diluted in SDS-PAGE loading buffer (25 mM tris-HCl, pH 6.6, 50 mM DTT, 1% SDS, 0.05% bromophenol blue and 5% glycerol) and samples were loaded on a 4-20% SDS-polyacrylamide gel. The proteins were transferred to a PVDF membrane (Bio-Rad, Wattford, UK) and detection was performed with a polyclonal rabbit anti-α-synuclein antibody 1:1500 (Chemicon, Temecula, Calif.) using the ECL+ chemiluminescent system (Amersham-Pharmacia, The Netherlands).

Cell Counting

We determined the number of α-synuclein positive cells in the striatum by counting every fifth 50μm section at a magnification of 40x. We considered the cells to be positive for α-synuclein if the object to be counted had darker chromagen staining than the surrounding background and could be clearly identified as a cell. We estimated the total number of positive cells per brain by multplication of the counts by 5 (Abercrombie, 1946).

Statistical Analysis

Data are presented as the mean ± SEM. The data were evaluated by a Kruskall-Wallis two-way analysis of variance. Data analysis was performed using SAS version 6.12 (SAS Institute Inc., 1997). All P-values are two-sided and considered statistically significant if they were <0.05.

Production of Lentiviral Vectors Systems with Reduced Immunoeenicity

An efficient production system for lentiviral vectors has been operational since 1996 (Naldini et al., 1996). The lentiviral vectors are efficient vehicula for gene transfer into non-dividing cells and likely candidates as vectors for future gene therapy. Although their widespread use, not much effort was undertaken to study potential immune response to vector preps. They are generally believed to be safe and without toxicity. Since we propose the use of LV to create disease models, especially models for neurodegenerative diseases that have a neuro-inflammatory component, we felt it important to study the immune response in full detail.

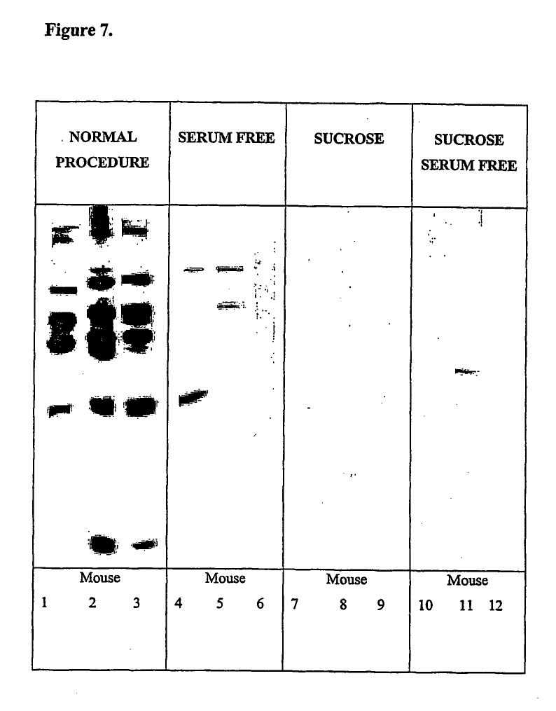

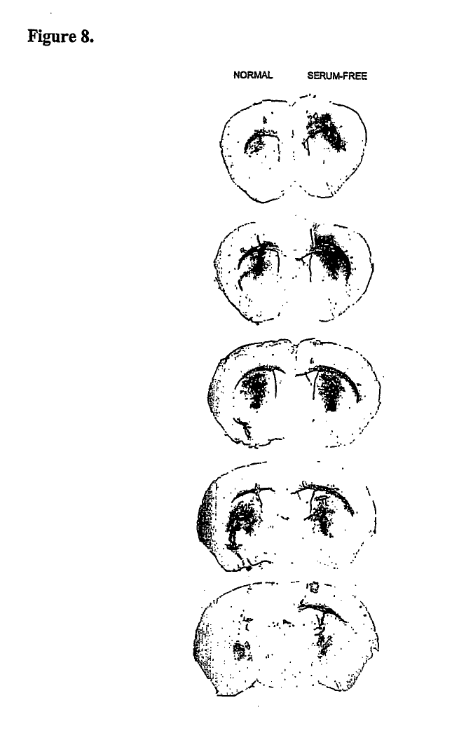

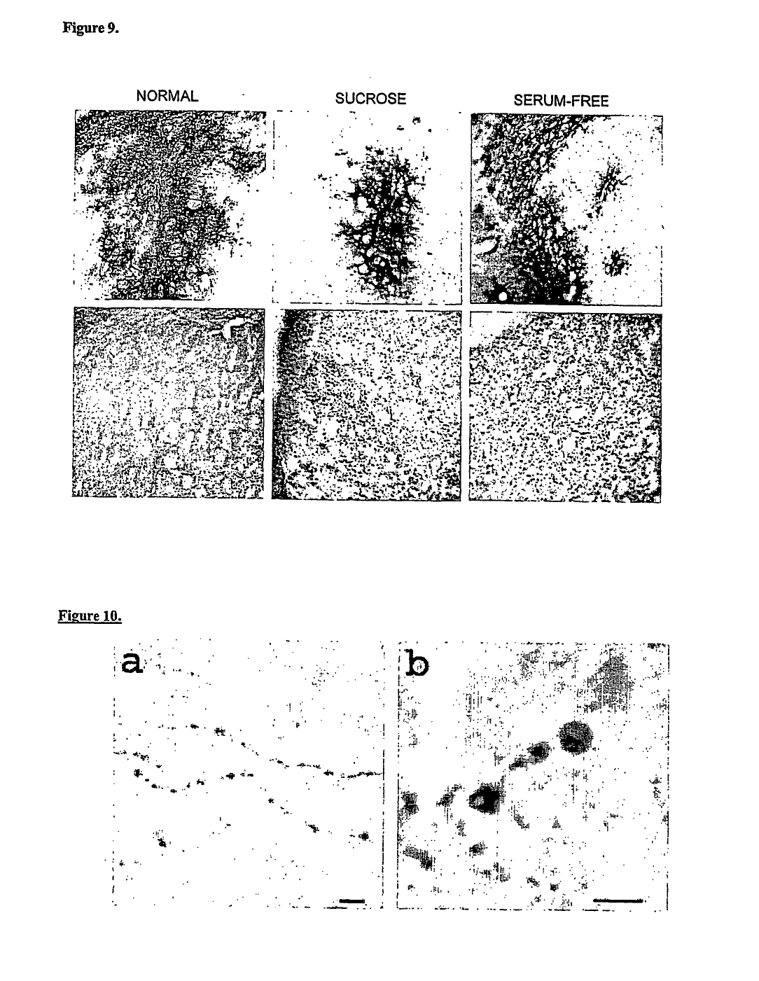

Although we demonstrated that lentiviral vectors are indeed efficient vehicula to deliver genes into animal brain, we have obtained evidence that injection of the current vector preparations that are made in the presence of fetal calf serum (FCS) and that are concentrated by ultracentrifugation (pelletting) is accompanied by a modest neuroinflammation. In WT mice but not in immune-deficient scid mice a transient decrease in NeuN expression is evident after lentiviral vector delivery (FIG. 9). Moreover, injected mice develop an immune response to vector proteins as evidenced by immunoblotting of vector preps with mouse serum (FIG. 7). We have also found that these antibodies are neutralising the vectors in cell culture (data not shown).

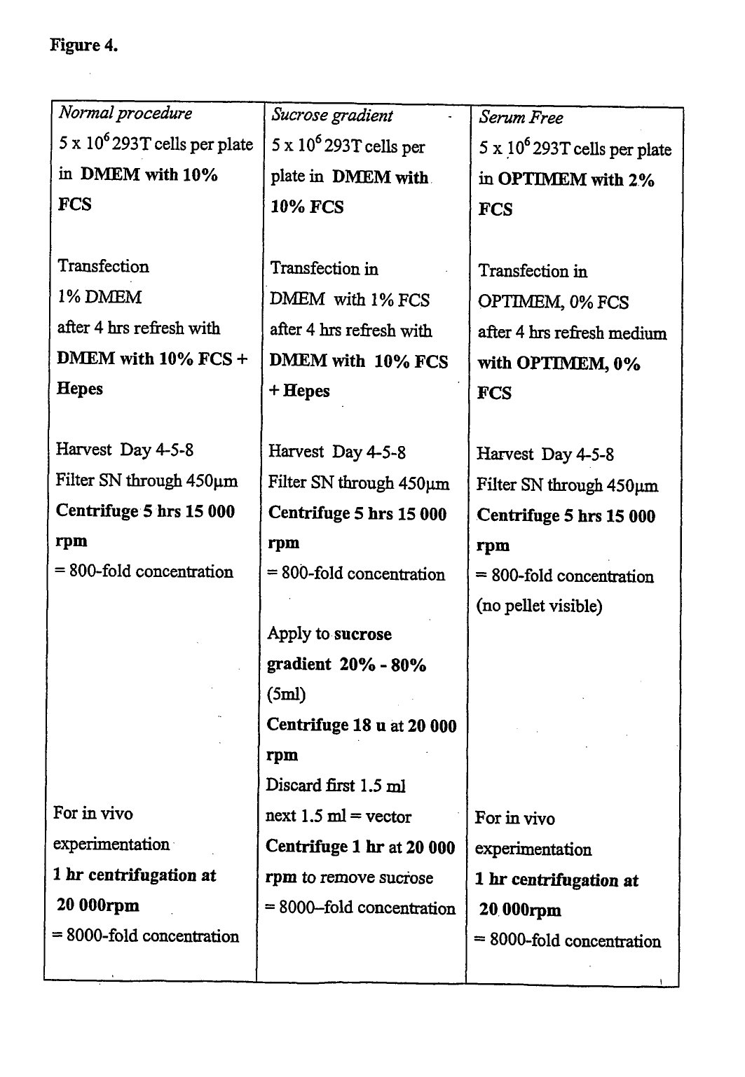

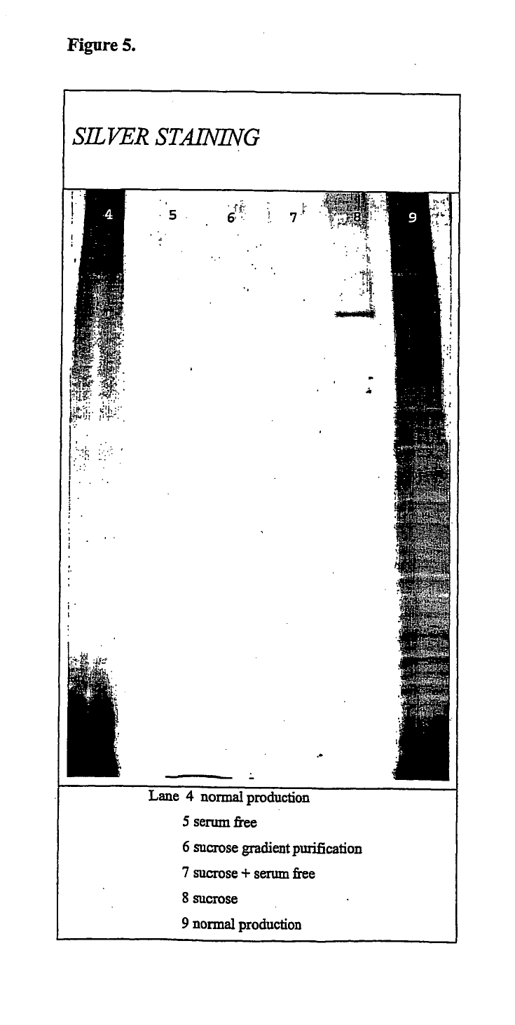

To avoid these unexpected immune reaction we optimised new vector, production protocols. In our new methodfor entiviral vector production serum is omitted FIG. 4, new protocol). As a result vector preps are pure as evidenced by silver staining, (FIG. 5) and a reduction in immune response (FIG. 7) and NeuN downregulation (FIG. 9) is seen.

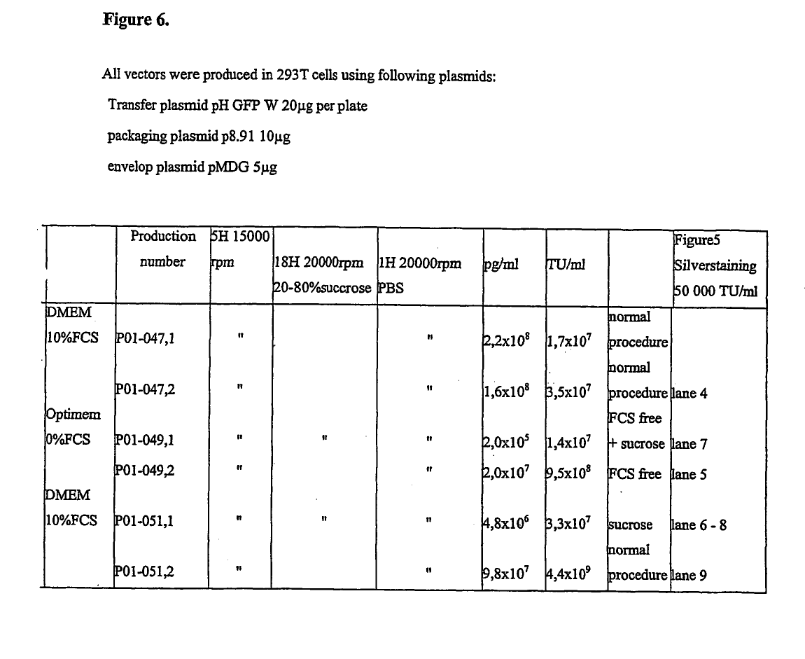

Tranduction efficiency of vectors is comparable to that of vectors produced by classical methods (FIG. 6, 8). After sucrose gradient ultracentrifugation, there is no immune response in mice (FIG. 7) but vector titers do decrease (FIG. 6).

Our new method allows the use of lentiviral vectors to create disease models by local overexpression of disease-associated genes for diseases wherein a neuroinflammatory component is important as is the case for neurodegenerative diseases. Purification should also be standard operating procedure for clinical application of lentiviral vectors.

### RESULTS

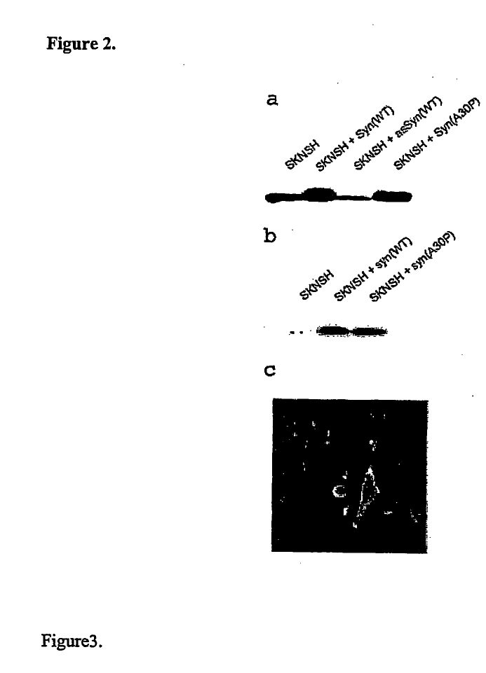

Lentiviral vectors mediate expression of α-synuclein in cell cultureThe expression of wild-type and mutant (A30P) α-synuclein from the lentiviral vector constructs, was confirmed by transfecting 293T (data not shown) and transducing SK-N-SH neuroblastoma cells and western blot analysis (FIG. 2a). Both wild type and mutant α-synuclein are expressed at high levels compared to the endogenous level. After transduction with vectors containing the antisense α-synuclein we observed a decrease in α-synuclein expression. Two stable SK-N-SH cell lines overexpressing respectively wild-type and (A30P) α-synuclein were selected after transduction with an α-synuclein-IRES-neo vector (FIG. 2b). Analysis of lysates of this stable cell lines by western blot (FIG. 2b) and immunocytochemistry (FIG. 2c) revealed expression of high levels of both wild type and mutant α-synuclein (FIG. 2c) compared to the endogenous levels (not shown).

Abnonnal Accumulation of α-synuclein in Cell Bodies and Neurites of Transduced Neurons

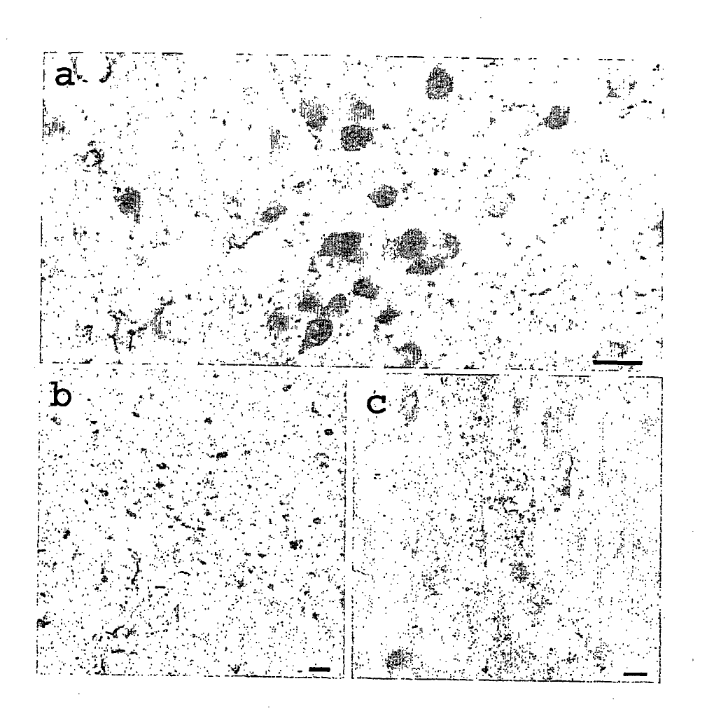

The normal distribution of endogenous mouse α-synuclein in the central nervous system is axonal and presynaptic. In contrast, two weeks after transduction high levels of both wild type and mutant α-synuclein were detected in the mouse neuronal cell bodies and neuritis. Most of the transduced cells have a neuronal phenotype (FIG. 3). Two weeks after injection of vectors encoding either wild type or mutant (A30P) α-synuclein in the mouse striatum few neurites in the cortex displayed α-synuclein immunoreactivity. After 6 months we observed strong inmunostaining of neuritic extensions in the cortex and small bulbous neurites (FIG. 10a). These α-synuclein containing bead like structures were separated by normal neuritic profiles (FIG. 10b). We detected no inclusions or beaded neurites in the contralateral hemisphere or in neurites from untransduced cells on the injected side (not shown). We did not observe major differences between animals injected with wild type or A30P mutated α-synuclein. Accumulation of wild type a-synuclein in the cell body and in neurites is however a characteristic feature of human brains with Lewy pathology[Fomo, 1996 #94; Baba, 1998 #107; Spillantini, 1997 #105]. Also bead like structures in neurites are one of the frequently observed neuritic changes in human brain pathology. The same vectors were also injected in rat brain. When a vector expressing wild type α-synuclein was injected in the substantia nigra of the rat, the same neuritic beads were observed in the striatum and in the cortex (not shown). Based on the knowledge of neuritic projections, this experiment shows us that these varicosities are located in the axons and are not due to the trauma at the side of injection. It also illustrates how brain pathology linked to gene expression can be induced by the same vector in different species.

Ubiguitin and α-synuclein Positive Cytoplasmic inclusion Bodies Appear in Aging Animals

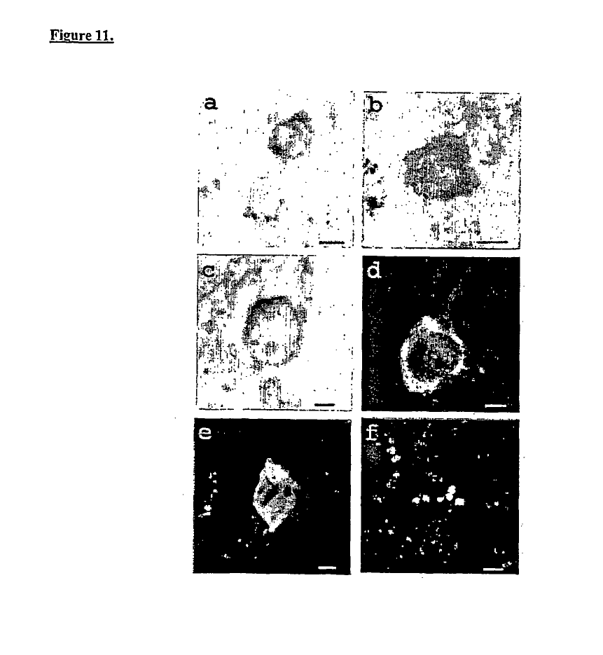

At 6 months post-injection, we detected α-synuclein-positive cytoplasmic inclusions in the α-synuclein positive neurons (FIG. 11a). At 10 months approximately 30% of the α-synuclein expressing cells contained small to large cytoplasmic inclusions. Most of those had a spherical morphology and the diameter was up to 6 μm (FIG. 11b). Some of the lesions had tangle-like shapes (FIG. 11c). No difference was observed between wild type and mutant α-synuclein. Lewy pathology in human brain is typically detected by ubiquitin immunostaining [Gai, 1995 #106; Forno, 1996 #94; Ince, 1998 #95]. To assess whether the inclusions were ubiquitin-positive, we performed double immunostainings with antibodies specific for human α-synuclein and ubiquitin and analyzed the sections using confocal microscopy. Approximately 60% of the α-synuclein-positive cells contained inclusions that stained for α-synuclein and ubiquitin at 6 months post injection (FIG. 11d). At 10-12 months, ubiquitin was also detected in the very large α-synuclein inclusions observed at that point in time (FIG. 11e) while some of the neuritic enlargements stained strongly for both α-synuclein and ubiquitin (FIG. 1f).

Long Term α-synuclein Expression Enduces Neurodeaeneration

A crucial feature of any in vivo model for PD is neurodegeneration. Age-dependent dopaminergic cell loss was seen in α-synuclein transgenic fruit flies but not in the currently available transgenic mice[Dawson, 2000 #58]. To quantify the number of α-synuclein-expressing cells after LV transduction, we performed cell counts at 2 weeks (n=6), 6 months (n=4) and 10-12 months (n32 3) after injection (FIG. 12). The total number of transduced cells at 2 weeks was set at 100%.

After 6 months the relative amount of α-synuclein expressing cells was 141±34%. This increase is not significantly different from the number of positive cells observed at two weeks after injection (P=0.33) and reflects probably the gradual accumulation of the α-synuclein protein. Thus, the expression levels of α-synuclein remain fairly constant for at least 6 months. These data are consistent with earlier reports of long-term expression of a GFP reporter genein mouse brain using lentiviral vectors[Blomer, 1997 #30] (Baekelandt et al., manuscript submitted). After 10-12 months only 55±11% α-synuclein-positive cells were observed. This was significantly lower than the number at 2 weeks (P<0.05) and the number at 6 months (P<0.05).

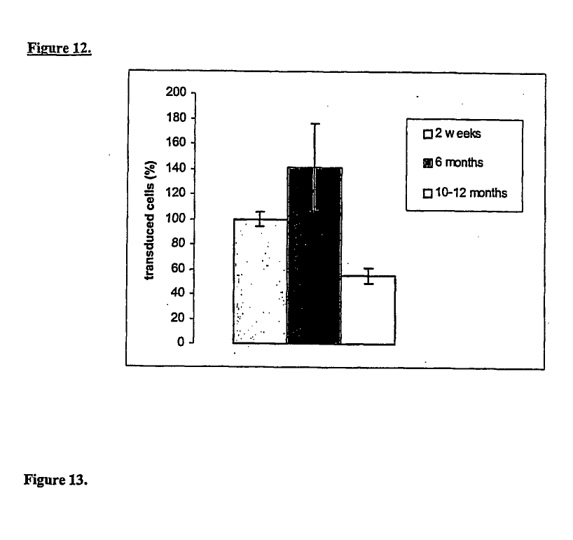

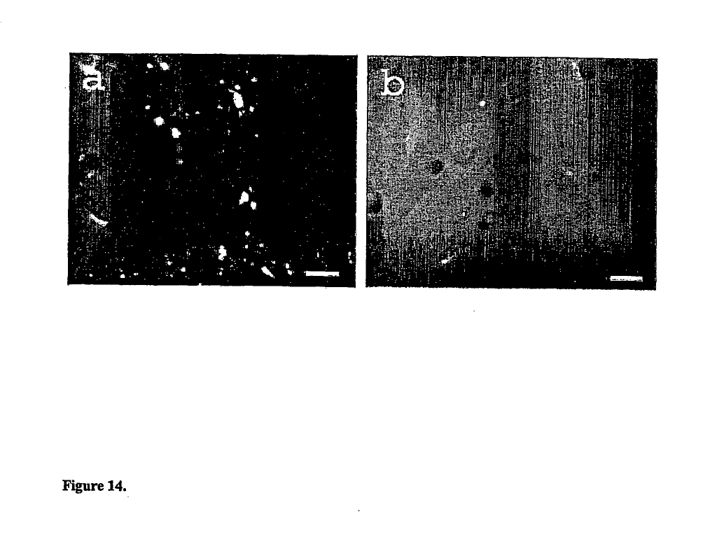

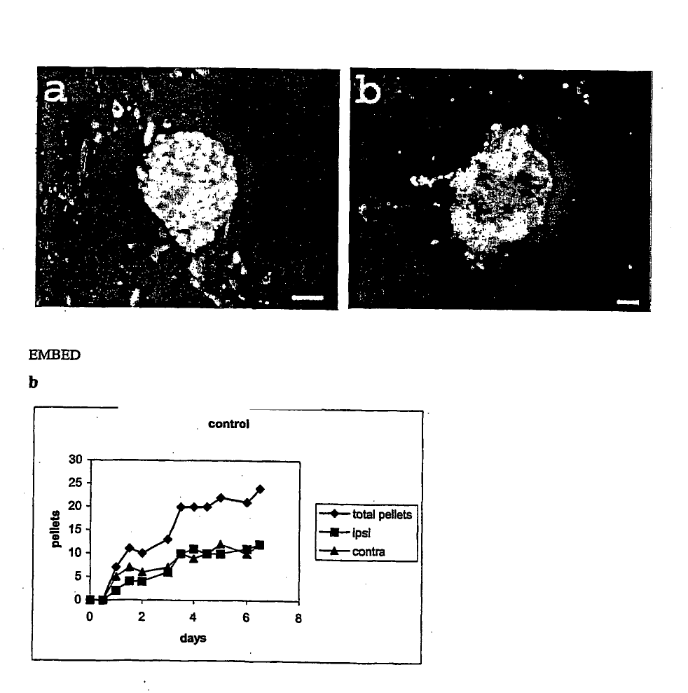

To determine if the decrease in α-synuclein expressing cells between 6 and 12 months could be a consequence of neuronal loss, we performed Fluoro-Jade B stainings. Fluoro-Jade B is a fluorescein derivative that selectively stains neurons undergoing neurodegeneration[Schmued, 1997 #109]. At 2 weeks after injection strong Fluoro-Jade B reactivity is observed both in brains injected with vectors encoding human WT or mutant α-synuclein or injected with control saline or defective LV particles (not shown). This is a consequence of the physical trauma induced by the injection of the needle. At 6 months there is little difference (not shown) and after 10 months we detected several Fluoro-Jade-B-positive neurons and fibers in the α-synuclein injected animals (FIG. 13a) but not after the control injections (FIG. 13b). Moreover, after double-staining of sections for α-synuclein and ubiquitin, several neurons with a degenerative morphology were detected from 6 months post injection (FIG. 14a, 14b). These data point to striatal neurodegeneration induced by chronic α-synuclein overexpression.

### SUMMARY OF THE INVENTION

A New Method for Locoregional Transgensis

The results obtained, pathological findings in neurons overexpressing a clinical mutant of α-synuclein, starting from two weeks after gene transfer, provide a new method to create disease models in animals that can be used to study pathogenesis, for target validation, for drug evaluation and for the development of new diagnostic tools. The model presented is a model for Parkinson's disease in mouse brain. The same method can be applied in other animals, in other organs and with genes associated with various diseases. Our method provides an alternative to classical transgenesis.

One embodiment of the present invention is thus overexpression of pathogenic proteins in selected and targeted tissues, preferably neural tissues, in non-human animals by stereotactic vector-mediated transfer of disease-associated genes into the brain (or another organ). If the gene transfer results in pathology reminiscent of the disease, a model has been created. In another embodiment by using antisense, transdominant negative mutant or ribozyme technology, proteins involved in pathogenetic pathways are downregulated.

A first preferred embodiment of present invention involves a method to create disease models in non-human animals using stereotactic and viral vector-mediated gene delivery. In a further preferred embodiment it involves a method to create disease models in non-human animals using stereotactic lentiviral vector mediated gene transfer. In a further preferred embodiment it involves a method to create disease models in non-human animals using stereotactic lentiviral vector mediated gene transfer in he brain of various species.

A second preferred embodiment of present invention involves a method to create disease model using stereotactic lentiviral vector mediated gene transfer in the brain of rodents (mice, rat).

A third preferred embodiment of present invention involves a method to create disease models in non-human animals using stereotactic lentiviral vector mediated gene transfer in the brain to overexpress or prevent expression of disease associated genes involved in neurodegeneratioin. More preferably it involves a method to create disease models in non-human animals usig stereotactic lentiviral vector mediated gene transfer in the brain to overexpress or prevent expression of disease associated genes involved in Parkinson's disease. Most preferably it involves a method to create disease models in non-human animals using stereotactic lentiviral vector mediated gene transfer of a polynucleotide sequence, an allelic variant or a homolog thereof, that encodes for α-synuclein or functional homologues thereof in the brain to overexpress α-synuclein, an isoform of α-synuclein or functional homologues thereof.

A fourth preferred embodiment of present invention involves a method to create locoregional, somatic transgenic non-human animals using vector-mediated gene transfer. More preferably it involves an animal model in mice for Parkinson's disease based on local, viral vector-mediated overexpression of wild type or mutant α-synuclein or functional derivatives thereof.

A fifth preferred embodiment of the present invention is a method to create a stable cell line that overexpresses WT or mutant α-synuclein that can be used to select compounds or genes that interfere with gene expression of α-synuclein or pathology induced by α-synuclein.

A sixth preferred embodiment is a method for reducing the immunogenicity of the lentiviral vector compositions, used for lentiviral mediated gene transfer in the brains of non-human animals. It also involves a lentiviral vector composition with normal transduction efficiency but avoid of immune reactions and more preferably a lentiviral vector composition with normal transduction efficiency but avoid of immune reactions in the brains of non-human animals. The invention thus also involves non immunogenic lentiviral vector preparations for creation of neurodegenerative disease models of animals by overexpressing of disease-associated genes in selected zones of the brains or selected neural tissues of non human animals without inducing neuroinflammation.

### REFERENCES TO THE APPLICATION

Abelibvich A, Schmitz Y. farinas I, Chol-Lundberg D, Ho WH, Castillo PE, Shinsky N, Verdugo JM, Armanini M, Ryan A, Hynes M, Phillips H, Sulzer D, Rosenthal A (2000) Mice lacking alpha-synuclein display functional deficits in the nigrostriatal dopamine system. Neuron 25:239-252.

Ausubel, F. M., Brent, R., Kingston, R. E., Moore, D. D., Seidman, J. G., Smith, J. A., Struhl, K. Eds. (1987). Current protocols in molecular biology. John Wiley & Sons New York.

Betarbet, R., Sherer, T. B., MacKenzie, G., Garcia-Osuna, M., Panov, A. V., Greenamyre, J. T. (2000). Chronic systemic pesticide exposure reproduces features of Parkinson's disease. Nat. Neurosci. 12, 1301-1306.

Burns RS, Chiueh CC, Markey SP, Ebert MH, Jacobowitz DM, Kopin IJ (1983) A primate model of parkinsonism: selective destruction of dopaminergic neurons in the pars compacta of the substantia nigra by N-methyl-4-phenyl-1,2,3,6-tetrahydropyridine. Proc Natl Academy Sci U S A 80:4546-4550.

Conway KA, Lee SJ, Rochet JC, Ding TT, Williamson RE, Lansbury PT, Jr. (2000) Acceleration of oligomerization, not fibrillization, is a shared property of both alpha-synuclein mutations linked to early-onset Parkinson's disease: implications for pathogenesis and therapy. Proc Natl Acad Sci USA 97:571-576.

Dawson TM (2000a) New animal models for Parkinson's disease. Cell 101:115-118.

Dawson VL (2000b) Neurobiology. Of flies and mice. Science 288:631-632.

Dull T, Zufferey R, Kelly M, Mandel RJ, Nguyen M, Trono D, Naldini L (1998) A third generation lentivirus vector with a conditional packaging system. J Virol 72:9873-9880.

Farrer M, Wtvrant-De Vrieze F; Crook R. Boles L, Perez-Tur J, Hardy J, Johnson WG, Steele J, Maraganore b, Gwinn K, Lynch T (1998) Low frequency of alpha-synuclein mutations in familial Parkinson's disease. Annals of Neurology 43:394-397.

Feany MB, Bender WW (2000) A Drosophila model of Parkinson's disease. Nature 404:394-398.

Gasser T, Muller-Myhsok B, Wszolek ZK, Oehlmann R, Calne DB, Bonifati V, Bereznai B, Fabrizio E, Vieregge P, Horstmann RD (1998) A susceptibility locus for Parkinson's disease maps to chromosome 2p13. Nature Genetics 18:262-265.

Haass C, Kahle PJ (2000) Parkinson's pathology in a fly [news; comment]. Nature 404:341,343.

Kitada T, Asakawa S, Hattori N, Matsumine H, Yamamura Y, Minoshima S, Yokochi M, Mizuno Y, Shimizu N (1998) Mutations in the parkin gene cause autosomal recessive juvenile parkinsonism. Nature 392:605-608.

Kruger R, Kuhn W, Muller T, Woitalla D, Graeber M, Kosel S, Przuntek H, Epplen JT, Schols L, Riess O (1998) Ala30Pro mutation in the gene encoding alpha-synuclein in Parkinson's disease. Nature Genetics 18:106-108.

Leroy E, Boyer R, Auburger G, Leube B, Uln G, Mezey E, Harta G, Brownstein MJ, Jonnalagada S, Chernova T, Dehejia A, Lavedan C, Gasser T, Steinbach PJ, Wilkinson KD, Polymeropoulos MH (1998) The ubiquitin pathway in Parkinson's disease. Nature 395:451-452.

Masliah E, Rockenstein E, Veinbergs I, Mallory M, Hashimoto M, Takeda A, Sagara Y, Sisk A, Mucke L (2000) Dopaminergic loss and inclusion body formation in alpha-synuclein mice: implications for neurodegenerative disorders. Science 287:1265-1269.

Polymeropoulos MH, Lavedan C, Leroy E, Ide SE, Dehejia A, Dutra A, Pike B, Root H, Rubenstein J. Boyer R, Stenroos ES, Chandraselchdarappa S Athianassiadou A, Papapetropoulos T, Johnson WG, Lazzarini AM, Duvoisin RC, Di Iorio G, Golbe LI, Nussbaum RL (1997) Mutation in the alpha-synuclein gene identified in families with Parkinson's disease. Science 276:2045-2047.

Sauer H, Oertel WH (1994) Progressive degeneration of nigrostriatal dopamine neurons following intrastriatal terminal lesions with 6-hydroxydopamine: a combined retrograde tracing and immunocytochemical study in the rat. Neuroscience 59:401-415.

Ueda K, Fukushima H, Masliah E, Xia Y, Iwai A, Yoshimoto M, Otero DA, Kondo J, Ihara Y, Saitoh T (1993) Molecular cloning of cDNA encoding an unrecognized component of amyloid in Alzheimer disease. Proc Natl Acad Sci U S A 90:11282-11286.

van der Putten H, Wiederhold KH, Probst A, Barbieri S, Mistl C, Danner S, Kaufftmann S, Hofele K, Spooren WP,Ruegg MA, Lin S, Caroni P, Sommer B, Tolnay M, Bilbe G: Neuropathology in mice expressing human α-synuclein. J Neurosci (2000) 20:6021-6019.

Zufferey R, Dull T, Mandel RJ, Bukovsky A, Quiroz D, Naldini L, Trono D (1998) Self-inactivating lentivirus vector for safe and efficient in vivo gene delivery. Journal of Virology 72:9873-9880.

We claim:

1. A method to create a neurological disease model in a non-human animal, wherein a transgene or transgenes are transferred by stereotactic and viral vector-mediated gene transfer in non-dividing cells or in terminally differentiated cells of said animal and result in a neuropathology.

2. The method of claim 1, wherein the transgene or transgenes express or overexpress in the non-dividing cells or in terminally differentiated cells of the brain.

3. The method of claim 1, wherein the transgene or transgens prevent the expression of a gene in the non-dividing cells or in terminally differentiated cells of the brain.

4. The method of any of the claims 1 to 3, wherein the neuropathology is an histological pathology.

5. The method of claim 4, wherein the histological pathology is visualised by histological stains, histochemical stains or immunohistochemical stains.

6. The method of claim 4, wherein the histological pathology is characterised by a technology of the group consisting of electron microscopy, transmission electron microscopy, phase-contrast microscopy, scanning electron microscopy, transmission microscopy and light microscopy.

7. The method of any of the claims 1 to 3, wherein the pathology is characterised by radiographic neuroimaging or by magnetic neuroimaging.

8. The method of any of the claims 1 to 3, wherein the pathology is characterised by computed tomography (CT).

9. The method of any of the claims 1 to 3, wherein the pathology is characterised by a neuroimaging method of the group consisting of magnetic resonance imaging (μMRI), 3D-MRI, gadolinium (Gd)-enhanced MRI and magnetic resonance spectroscopic imaging (MRSI) and microscopic magnetic resonance Imaging (μMRI).

10. The method of any of the claims 1 to 9, wherein the stereotactic and viral vector-mediated gene transfer is a stereotactic lentiviral vector mediated gene transfer.

11. The method of any of the claims 1 to 10, wherein the stereotactic and viral vector-mediated gene transfer is a stereotactic leitiviral vector mediated gene transfer in the brain of rodents.

12. The method of any the claims 1 to 11, wherein the stereotactic and viral vector-mediated gene transfer is a stereotactic lentiviral vector mediated gene transfer in the brain to overexpress or prevent expression of genes involved in neurodegeneration.

13. The method any of the claims 1 to 12, wherein the stereotactic and viral vector-mediated gene transfer is a stereotactic lentiviral vector mediated gene transfer in the brain to overexpress or prevent expression of genes involved in Parkinson's disease.

14. The method any of the claims 1 to 13, wherein the stereotactic and viral vector-mediated gene transfer is a stereotactic lentiviral vector mediated gene transfer in the brain to over-express wild type or mutant α-synuclein or functional derivatives thereof.

15. The method any of the claims 1 to 14, wherein the transgenes comprise the OLsynuclein gene an allelic variant, minigene, a homolog thereof or A30P, that encode for α-synuclein, an isoform of α-synuclein or functional homologues thereof or at least a portion thereof, said transgene obtainable by a method comprising 1) producing HIV-1-derived vector particles, pseudotyped with the envelope of non related virus, said HIV-1 derived vector particles obtainable by tranfecting suitable cells in suitable agents with a suitable packaging plasmid encoding viral gag and pol proteins, a plasmid encoding the envelope of a non related virus and a plasmid encoding α-synuclein gene or an allelic variant, minigene or a homolog thereof which is flanked by LTR's, 2) isolating and concentrating the vector particles 3) redisolving the vector particles in a suitable agent, 4) injecting the vector particle solution in stereotactically defined targets of an non-human animal brain.

16. The method any of the claims 1 to 15, wherein the transgenes comprise the α-synuclein gene an allelic variant, minigene, a homolog thereof or A30P, that encode for α-synuclein, an isoform of α-synuclein or functional homologues thereof or at least a portion thereof, said transgene obtainable by a method comprising producing HIV-1-derived vector particles, pseudotyped with the envelope of non related virus, said HIV-1 derived vector particles obtainable by tranfecting suitable cells in suitable agents with a suitable packaging plasmid encoding viral gag and pol proteins, a plasmid encoding the envelope of a non related virus and the a-synuclein encoding -plasid, pHMWS-SYN(α-synuclein-gene) 1) a derivative of pHR′2) isolating the vector particles 3) redisolving the vector particles in a suitable agent, 4) injecting the vector particle solution in sterotactically defined targets of an non-human animal brain 5).

17. The method any of the claims 1 to 16, further comprising the step of histochemical analysis of expression of transgene and induction of pathology.

18. The method any of the claims 1 to 16, further comprising the step of analysis of neurodegeneration by cell counting or Fluorojade staining.

19. Any of claims 1 to 18, wherein the vector particles are downstream processed or purified to a purity level of reduced immunogenicty or to a purity level whereby said vector particles do not induce or evoke an immune or inflammatory response.

20. Any of the claims 1 to 19, wherein the vector particles are downstream processed or purified to a purity level of reduced immunogenicity or to a purity level whereby said vector particles do not induce or evoke an immune response or inflammatory at a dose of efficient transfer of transgenes into the animal brain.

21. The claims 19 or 20, wherein the vector particles are purified or downstream processed by a chromatography.

22. Any of the claims 1 to 21, wherein the vector particles are obtainable form transfected cell which were cultured in a serum free medium.

23. Any of the claims 1 to 22, wherein gene transfer is mediated by a lentiviral vector system that is serum free.

24. Any of the claims 1 to 23, wherein gene transfer is mediated by a lentiviral vector system that is free of proteins.

25. Any of the claims 1 to 24, characterised in that the lentiviral vector system does not evoke an immune response.

26. The method of any of the claims 1 to 25, to create locoregional somatic transgenic non-human animals.

27. The method of any of the claims 1 to 26, to create locoregional somatic transgenic rodents.

28. The method of any of the claims 1 to 27, to create locoregional somatic transgenic mice.

29. The method of any of the claims 1 to 2, to create locoregional somatic transgenic rat.

30. A locoregional, somatic transgenic non-human animal created by stereotactic and viral vector-mediated mediated gene transfer in non-dividing cells or in terminally differentiated cells of said animal resulting in a neuropathology.

31. The animal of claim 30 created by stereotactic and viral vector-mediated mediated gene transfer in in non-dividing cells or in terminally differentiated cells of the brain to overexpress locoregional or prevent expression locoregional of genes involved in neurodegeneration.

32. The animal of claim 30 or 31, wherein the disease associated gene is involved in Parkinson's disease.

33. The animal of claim 30, 31 or 32, wherein stereotactic and viral vector-mediated mediated transgene transferred in non-dividing cells or in terminally differentiated cells of the brain locoregional overexpresses wild type or mutant α-synuclein or functional derivatives thereof.

34. The animal of any of the claims 30 to 33, wherein stereotactic and viral vector-mediated mediated transgene transferred in non-dividing cells or in terminally differentiated cells prevents locoregional expression of wild type or mutant α-synuclein.

35. The animal of any of the claims 30 to 34, wherein said animal develops a macroscopic or microscopic neuropathology.

36. The animal of any of the claims 30 to 35, said animal being a rondent.

37. The animal of any of the claims 30 to 36, harboring in their neural tissue or brains a polynucleotide sequence, an allelic variant or homolog thereof, that encodes for wild type or mutant α-synuclein or functional derivatives or homologues thereof and overexpresses -synuclein, isoform of α-synuclein or functional homologues thereof locoregional in said neural tissue or said brain.

38. The animal of any of the claims 30 to 37, wherein said animal is a model for a neurodegenrative disease.

39. The animal of any of the claims 30 to 38, wherein said animal is a model for Parkinson's disease.

40. The animal model of any of the claims 30 to 39, created by a method of any of the claims 1 to 29.

41. The use of animal model of claim 38, 39 or 40, for screening a plurality of compounds to identify a compound that prevents or inhibits the development of neurpathology, which comprises treating of the animal model with said compound or compounds and determining the neuropathologies by means of histological, radiographic or magnetic neuroimaging.

42. The use of animal model of claim 38, 39 or 40, for monitoring the effect of therapy administered to a mammal having a neuropathology comprising 1) histological, radiographic or magnetic neuroimaging and 2) comparing the abundance of each chosen feature in neuroimages with the abundance of that chosen feature in the neuroimages of control animals free of said neuropathoplogy.

43. A cell line that contains the α-synuclein gene, an allelic variant, minigene, a homolog thereof or A30P, to over-express α-synuclein, an isoform of α-synuclein or functional homologues thereof or at least a portion thereof, obtainable by a method comprising producing HIV-1-derived vector particles, pseudotyped with the envelope of non related virus, said HIV-1 derived vector particles obtainable by tranfecting suitable cells in suitable agents with a suitable packaging plasmid encoding viral gag and pol proteins, a plasmid encoding the envelope of a non related virus and the α-synuclein encoding plasmid, pHMWS-SYN(α-synuclein-gene)IRES-NEO 1) a derivative of pHR′ 2) isolating the vector particles 3) redisolving the vector particles in a suitable agent 4) transducing the cell line with the vector 5) selecting expressors by passaging cells in the presence of geneticin.

44. The cell line described in claim 43 derived from a SKNSH cell line.

45. The use of cell lines described in claim 42 or 43 for testing pharmaceutical compounds, pharmaceutical compositions or nucleic acids that interfere with α-synuclein expression or α-synuclein-induced pathology.

46. Mediated gene transfer into non human animal brains or human brains by a lentiviral vector system that is protein free and does not evoke an immune response.
